(19) United States
(12) Patent Application Publication
(10) Pub. No.: US 2009/0149384 A1
(43) Pub. Date: Jun. 11, 2009

(54) Protection of Photoreceptors in Experimental Autoimmune Uveitis

(75) Inventors: Narsing A. Rao, San Marino, CA (US); Sindhu Saraswathy, Burbank, CA (US)

Correspondence Address: MCDERMOTT WILL & EMERY LLP, 28 STATE STREET, BOSTON, MA, 02109-1775, US

(73) Assignee: Doheny Eye Institute, Los Angeles, CA (US)

(21) Appl. No.: 12/330,920

(22) Filed: Dec. 9, 2008

Related U.S. Application Data
(60) Provisional application No. 61/012,598, filed on Dec. 10, 2007.

(51) Int. Cl. A61K 38/17; A61P 27/02

(52) U.S. Cl. 514/12

(57) ABSTRACT

Techniques are described for the administration of crystallins, e.g., αA and/or β crystallin, to protect retinal photoreceptors of subjects inoculated with Experimental Autoimmune Uveitis. The present disclosure provides a unique and novel approach to the prevention of photoreceptor degeneration in uveitis and other blinding diseases mediated by oxidative stress including retinitis pigmentosa, macular degeneration, diabetic retinopathy and glaucoma through the administration of crystalline, e.g., αA and/or β crystallin.

### RELATED APPLICATION

This application claims the benefit of U.S. Provisional Patent Application Ser. No. 61/012,598 filed 10 Dec. 2007, the entire content of which is incorporated herein by reference.

### BACKGROUND

Photoreceptor degeneration mediated by oxidative stress is a common factor in uveitis and other blinding diseases including retinitis pigmentosa, macular degeneration, diabetic retinopathy and glaucoma. Experimental autoimmune uveitis (“EAU”) is an animal model that closely resembles human uveitis, by injecting S-antigen and inter-photoreceptor retinoid-binding protein (“IRBP”) in rats and mice respectively.

Uveitis specifically refers to inflammation of the middle layer of the eye, termed the “uvea” but in common usage may refer to any inflammatory process involving the interior of the eye, with inflammation specifically of the uvea termed iridocyclitis. Uveitis is estimated to be responsible for approximately 10% of the blindness in the United States. Uveitis requires an urgent referral and thorough examination by an ophthalmologist, along with urgent treatment to control the inflammation. Symptoms of uveitis can include: redness of the eye, blurred vision, sensitivity to light (photophobia); dark, floating spots along the visual field; and, eye pain.

The prognosis is generally good for those who receive prompt diagnosis and treatment, but serious complication (including cataracts, glaucoma, band keratopathy, retinal edema and permanent vision loss) may result if left untreated. The type of uveitis, as well as its severity, duration, and responsiveness to treatment or any associated illnesses, all factor in to the outlook.

Uveitis is typically treated with glucocorticoid steroids, either as topical eye drops (prednisolone acetate) or oral therapy with prednisolone tablets. In addition topical cycloplegics, such as atropine or homatropine, may be used. Antimetabolite medications, such as methotrexate are often used for recalcitrant or more aggressive cases of uveitis.

While such prior art treatments and techniques can lead to satisfactory results, often they do not. Accordingly, what is desirable are improved techniques for treating uveitis.

### SUMMARY

The present disclosure is directed to improved techniques for treating uveitis by administration of small heat shock proteins, e.g., crystallin, to the treatment subject. Embodiments of the present disclosure address the shortcomings noted previously for prior art techniques uveitis treatment techniques. Embodiments have shown protection against uveitis animals. Embodiments may provide similar protections against uveitis in humans.

Aspects of the present disclosure provide methods of treating and protecting photoreceptor layers with crystallin by intravenous injection. Exemplary embodiments can utilize αA and/or β crystallin.

An embodiment can include a method for treating uveitis in a subject, including administering to the subject an effective therapeutic amount of a therapeutic agent formulated with a suitable pharmaceutical carrier, wherein the therapeutic agent is a crystallin protein, or fragment thereof. The crystallin protein, or fragment thereof, can be selected from the group consisting of α crystallin, β crystalline and a combination thereof. The subject is human in exemplary embodiments. The administration can be by way of intravenous injection, intramuscular injection, intraperitoneal injection, oral, subcutaneous, sublingual, and combinations thereof.

The crystalline protein, or fragment thereof, is αA crystallin or β crystallin in exemplary embodiments. The suitable carrier can be saline. The effective therapeutic amount can range from about 1 μg to about 20 μg of crystallin, or fragment thereof, in exemplary embodiments/applications. The method can be used for the treatment of retinitis pigmentosa, macular degeneration, diabetic retinopathy and/or glaucoma.

The inventors have conducted research, according to embodiments of the present disclosure, showing marked upregulation of more than 30 fold in the gene expression of αA crystallin, β crystallin and γ crystallins in the retina of early EAU during day 7 post immunization with IRBP in B10RIII mice. The protein expression of αA crystallin was also increased 10 fold in the retina in early EAU compared to the controls. This increase was localized primarily in the photoreceptor inner segments, the site of mitochondrial oxidative stress. Moreover, αA-crystallin could inhibit the apoptotic process effectively, in both the in vitro and in vivo assays, as revealed by αA-crystallin knockout mice during early EAU. There was more inflammation and apoptosis in αA knockout mice compared to the wild-type animals. These observations indicate that upregulation of αA affords a protective mechanism, directed against photoreceptor mitochondrial oxidative stress in early EAU. Research of the inventors has assessed whether intravenous injection of αA, β and γ crystalline into EAU animals would protect the photoreceptors.

Other features and advantages of the present disclosure will be understood upon reading and understanding the detailed description of exemplary embodiments, described herein, in conjunction with reference to the drawings.

### BRIEF DESCRIPTION OF THE DRAWINGS

Aspects of the disclosure may be more fully understood from the following description when read together with the accompanying drawings, which are to be regarded as illustrative in nature, and not as limiting. The drawings are not necessarily to scale, emphasis instead placed on the principles of the disclosure. In the drawings:

FIG. 1 shows photomicrographs (A)-(D) of retinal tissue of B10RIII mice induced with EAU after 21 days of treatment, respectively, with saline solution (placebo), αA crystalline, β crystalline, and γ crystalline;

FIG. 2 is an enlargement of photomicrograph A of FIG. 1;

FIG. 3 is an enlargement of photomicrograph B of FIG. 1;

FIG. 4 is an enlargement of photomicrograph C of FIG. 1;

FIG. 5 is an enlargement of photomicrograph D of FIG. 1; and

FIG. 6 is a box diagram representing a method according to an exemplary embodiment of the present disclosure.

While certain embodiments depicted in the drawings, one skilled in the art will appreciate that the embodiments depicted are illustrative and that variations of those shown, as well as other embodiments described herein, may be envisioned and practiced within the scope of the present disclosure.

### DETAILED DESCRIPTION

The present invention and disclosure are directed to techniques providing for the protection of photoreceptors by treatment with small heat shock proteins. Exemplary embodiments can provide treatment of Experimental Autoimmune Uveitis (“EAU”) by small heat shock proteins or crystallins. Experimental autoimmune uveitis is an animal model that closely resembles human uveitis, by injecting uveotogenic-antigen in animal models such as rats and mice. Embodiments of the present disclosure may provide similar protection against uveitis and other similar eyes diseases/symptoms in humans. Exemplary embodiments of the present disclosure can utilize αA and/or β Crystallin for uveitis and/or EAU.

Crystallins can be used for uveitis treatment according to embodiments of the present disclosure. Suitable crystallins can include but are not limited to αA and/or β Crystallin. So-called α-Crystallins were originally recognized as proteins contributing to the transparency of the mammalian eye lens. Most members of the diverse -crystallin family have four common structural and functional features: (i) a small monomeric molecular mass between 12 and 43 kDa; (ii) the formation of large oligomeric complexes; (iii) the presence of a moderately conserved central region, the so-called -crystallin domain; and (iv) molecular chaperone activity. Since -crystallins are induced by a temperature upshift in many organisms, they are often referred to as small heat shock proteins (sHsps) or, more accurately, -Hsps. Beta-Crystallins are polydisperse, oligomeric structural proteins that play a major role in forming the high refractive index of the eye lens.

### Methods:

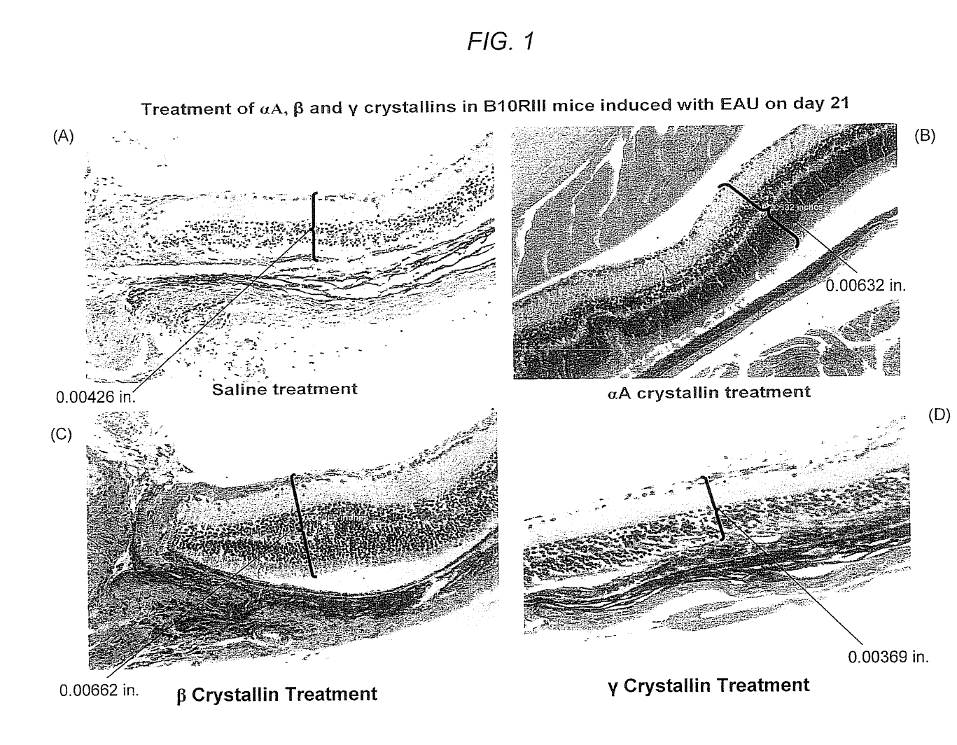

For an embodiment of the disclosure concerning EAU, EAU was induced in twenty four 8 week old B10RIII mice with IRBP. Onset of the disease in these animals occurred on day 10 and peaked on day 14. Six animals each were treated with 10 μg of recombinant αA, β and γ crystallin administered intravenously. EAU mice were injected intravenously every second day from day 12 onwards till day 20. EAU animals treated with saline served as control groups. Mice were sacrificed on day 21 and fixed in formalin. Paraffin sections (5μm) from these eyes were processed for histological analysis by H & E staining. The retinal thickness from each eye at the juxtapapillary, equator, and ora serrata areas were measured using light microscope, e.g., as shown collectively in FIG. 1 and displayed in Tables 1-2, infra.

FIG. 1 shows photomicrographs (A)-(D) of retinal tissue of B10RIII mice induced with EAU after 21 days of treatment, respectively, with saline solution (placebo), αA crystalline, β crystalline, and γ crystalline. As shown in micrograph (A) of FIG. 1, the retinal thickness resulting from the saline treatment was about 0.00426 in. As shown in micrograph (B) of FIG. 1, the retinal thickness resulting from the αA crystalline treatment was about 0.00632 in. As shown in micrograph (C) of FIG. 1, the retinal thickness resulting from the β crystalline treatment was about 0.00662 in. And, as shown in micrograph (D) of FIG. 1, the retinal thickness resulting from the and γ crystalline treatment was about 0.00369 in.

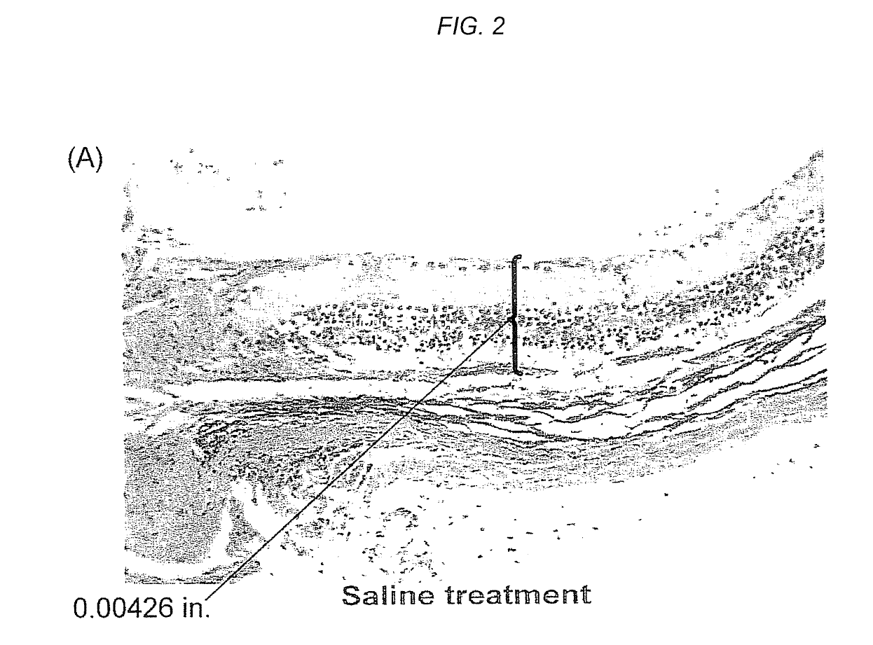

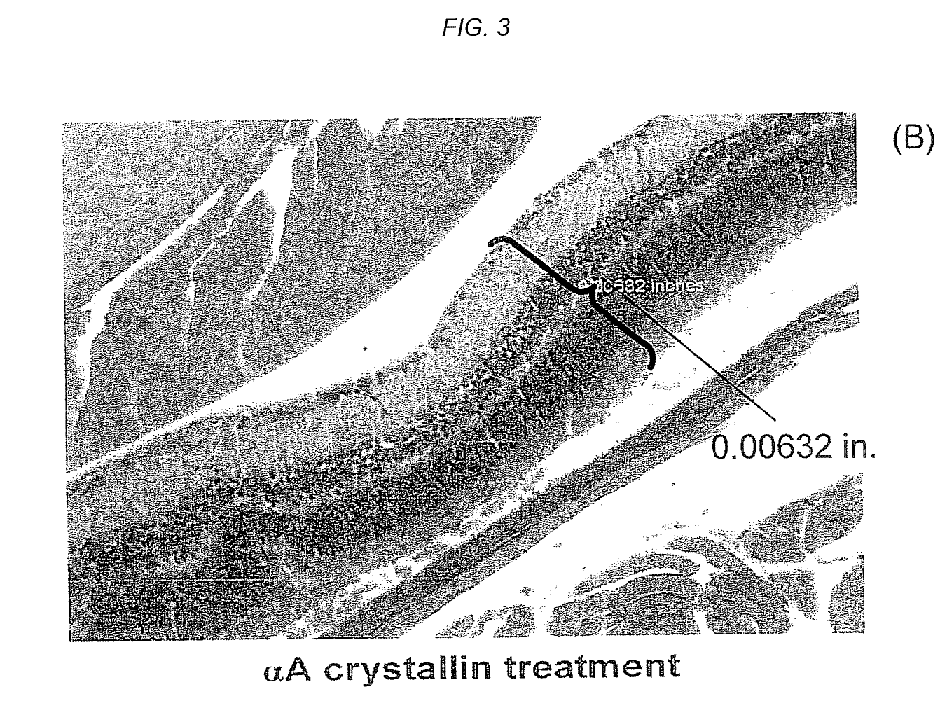

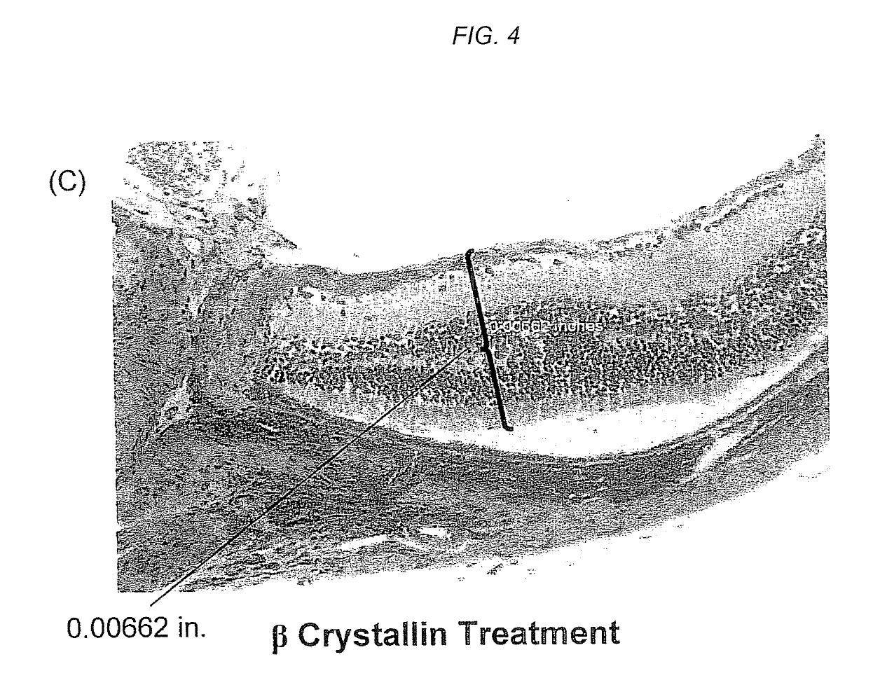

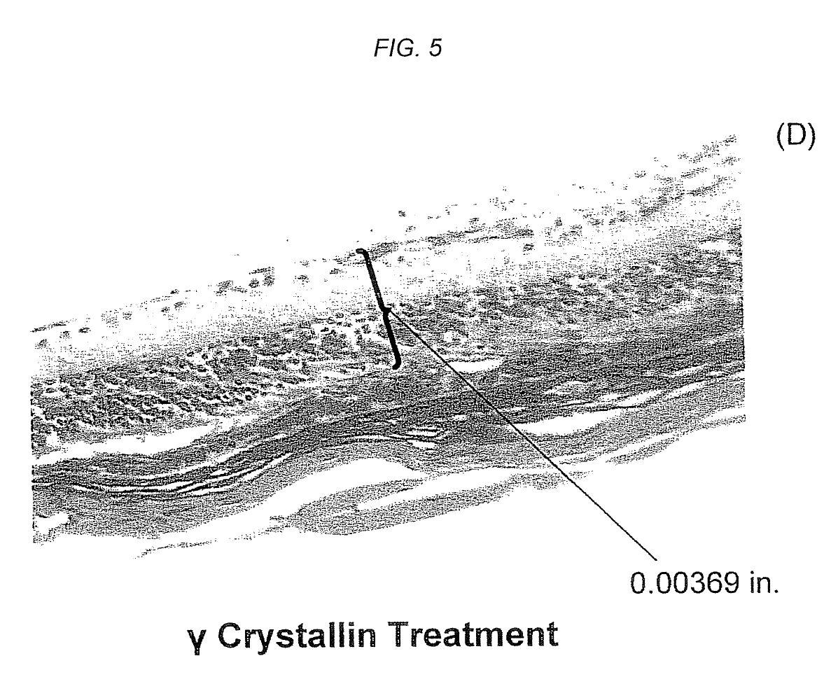

FIG. 2 is an enlargement of photomicrograph A of FIG. 1; FIG. 3 is an enlargement of photomicrograph B of FIG. 1; FIG. 4 is an enlargement of photomicrograph C of FIG. 1; and FIG. 5 is an enlargement of photomicrograph D of FIG. 1; and

Statistical analysis: Data are presented, supra, in Tables 1 and 2 as means±standard error of mean (“s.e.m.”). The retinal thickness at different points were analyzed by one way analysis of variance (“ANOVA”) using the standard Tukey-Kramer multiple comparisons test.

Results: Histology revealed destruction of photoreceptor layers in the EAU animals treated with saline, whereas in the EAU mice treated with αA crystallin and β crystallin, the photoreceptors were preserved on day 21. The photoreceptors were also destroyed in the animals treated with γ crystallin and looked similar to the controls groups. The retinal thickness of each eye was measured at the juxtapapillary, equator and ora serrata areas of all the eyes. See, e.g., FIG. 1.

Statistical analysis was performed by ANOVA using Tukey Kramer multiple comparison test. Results showed that the retinal thickness in αA crystallin and β crystallin treated animals were significantly greater than the control animals injected with saline and those injected with γ crystallin in all the areas of the retina studied. (P<0.001).

There was no significant difference in the retinal thickness between αA and β crystallin treated animals (P>0.05), whereas there was a significant difference between the αA and γ crystallin and between β and γ crystalline treatment groups. (P<0.01). See Tables 1 and 2 supra and FIGS. 1-5.

TABLE 1
showing the retinal thickness in microns from the Juxtapapillary, Equator and
Ora Serrata areas of EAU mice treated with αA, β and γ crystallins from day 12 to day 20
post immunization.

| Treatment | | | | | | |
|---|---|---|---|---|---|---|
| groups | Juxtapapillary area | | Equator area | | Ora Serrata area | |
| EAU with | Mean ± s.e.m | Median | Mean ± s.e.m | Median | Mean ± s.e.m | Median |
| Saline | 141.90 ± 9.53 | 141.05 | 119.24 ± 6.72 | 119.33 | 86.15 ± 2.63 | 86.18 |
| EAU with αA | 195.42 ± 5.82 | 203.20 | 179.09 ± 9.36 | 181.74 | 112.64 ± 6.76 | 108.20 |
| crystallin | | | | | | |
| EAU with β | 192.11 ± 10.58 | 201.55 | 165.47 ± 10.23 | 170.51 | 107.20 ± 5.75 | 108.84 |
| crystallin | | | | | | |
| EAU with γ | 146.78 ± 9.92 | 0138.17 | 119.99 ± 6.33 | 122.56 | 87.64 ± 3.32 | 90.30 |
| crystallin | | | | | | |

TABLE 2
Comparison between different treatment groups showing P value

| Comparison between different | | | Ora Serrata |
|---|---|---|---|
| treatment groups | Junta Papillary area | Equator area | area |
| Saline vs αA crystallin treatment | ** P < 0.01 | ***P < 0.001 | **P < 0.01 |
| Saline vs β crystallin treatment | ** P < 0A1 | ** P < 0.01 | *P < 0.05 |
| Saline vs γ crystallin treatment | Ns P > 0.05 | Ns P > 0.05 | Ns P > 0.05 |
| αA crystallin treatment vs β crystallin | Ns P > 0.05 | Ns P > 0.05 | Ns P > 0.05 |
| treatment | | | |
| αA crystallin treatment vs γ crystallin | ** P < 0.01 | **″ P < 0.001 | ** P < 0.01 |
| treatment | | | |
| β crystallin treatment vs γ crystalline | ** P < 0.01 | ** P < 0.01 | * P < 0.05 |
| treatment | | | |

### Description of Technology

The inventors' research has shown marked upregulation of more than 30 fold in the gene expression of αA crystalline, β crystallin and γ crystallins in the retina of early EAU during day seven post immunization with IRBP in B10RIII mice. The protein expression of αA crystallin was increased 10 fold in the retina in early EAU compared to the controls. This increase was localized primarily in the photoreceptor inner segments, the site of mitochondrial oxidative stress.

Moreover, αA-crystallin was observed to inhibit the apoptotic process effectively, in both the in vitro and in vivo assays, as revealed by αA-crystallin knockout mice during early EAU. There was more inflammation and apoptosis in αA knockout mice compared to the wildtype animals. These observations suggest that upregulation of αA affords a protective mechanism, directed against photoreceptor mitochondrial oxidative stress in early EAU. In subsequent research by the present inventors an assessment was made as to whether intravenous injection of αA, β and γ crystallin into EAU animals would protect the photoreceptors. By extension, the results of such research are believed to be applicable to humans.

### Methods and Results:

As noted previously, EAU was induced in four groups of B10RIII mice with IRBP. Each group of animals were treated intravenously with 10 μg of recombinant αA, β and γ crystallin respectively. EAU animals treated with saline served as control groups. EAU mice were injected intravenously every second day from day 12 onwards till day 20. Mice were sacrificed on day 21 and the eyes were fixed in formalin. Paraffin section (5 μm) from these eyes were processed for histological analysis by H & E staining.

Histology revealed destruction of photoreceptor layers in the EAU animals treated with saline, whereas in the EAU mice treated with αA crystallin and β crystallin, the photoreceptors were preserved on day 21. See FIG. 1. The photoreceptors were destroyed in the animals treated with γ crystallin and looked similar to the controls groups. See FIG. 1.

The retinal thickness of each eye was measured at the juxtapapillary, equator and ore serrata areas of all the animals. Statistical analysis was performed by ANOVA using Tukey Kramer multiple comparison test. Results showed that the retinal thickness in αA, and β crystallin treated animals were significantly more than the control animals injected with saline and γ crystallin in all the areas of the retina studied. (P<0.001). See Tables 1-2. See FIG. 1, with noted retinal thicknesses of 0.00426 in for saline treatment, 0.00632 in. for αA crystalline treatment, 0.00662 in. for β crystalline treatment, and 0.00369 in for γ crystalline treatment.

The results suggest to the present inventors that administration of αA and β crystallin offers a unique and novel approach to the prevention of photoreceptor degeneration in uveitis and other blinding diseases mediated by oxidative stress including retinitis pigmentosa, macular degeneration, diabetic retinopathy and glaucoma. The results may also be valid for other members of the small heat shock protein family such as crystallin forms, including other α crystallins, e.g., αB crystallin.

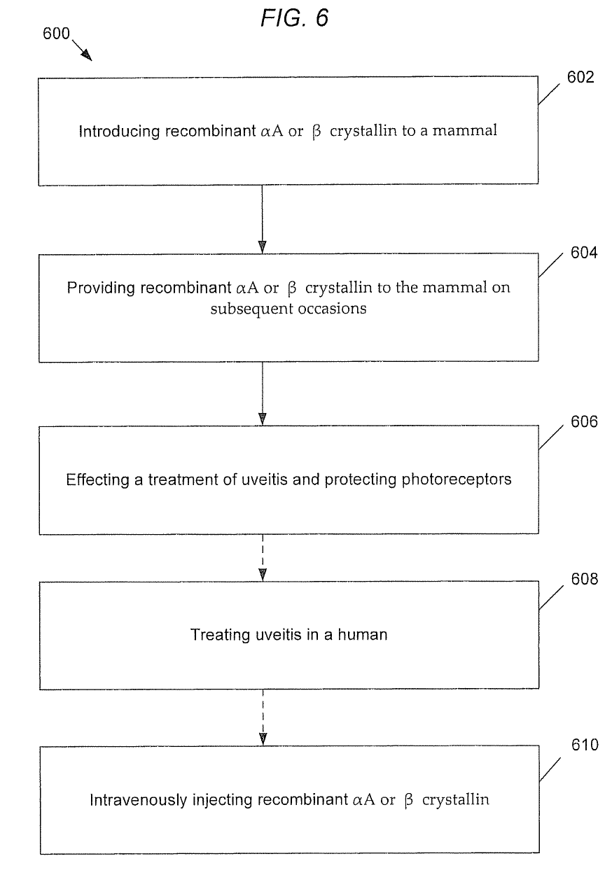

FIG. 6 is a box diagram representing a method according to an exemplary embodiment of the present disclosure. As shown, αA or β crystallin can be introduced to a mammal (or animal), as described at 602. Subsequent applications (doses) of αA or β crystallin may be made to the mammal, as described at 604. In exemplary embodiments, subsequent administrations of 20 μg may be made intravenously to a mammal on the 12th day after an initial administration and further on every second day through day 20.

Continuing with the description of method 600, photoreceptors of the eye may be protected, as described at 606, in the treatment of uveitis. Exemplary embodiments may include introduction of crystallin to a human, as described at 608. Exemplary embodiments may utilize intravenous injection, as described at 610. Other embodiments may utilize intramuscular injection, intraperitoneal injection, oral, subcutaneous, sublingual, and/or combinations thereof

While certain embodiments have been described herein, it will be understood by one skilled in the art that the methods and treatments of the present disclosure may be embodied in other specific forms without departing from the spirit thereof.

Accordingly, the embodiments described herein, and as claimed in the attached claims, are to be considered in all respects as illustrative of the present disclosure and not restrictive.

What is claimed is:

1. A method for treating uveitis in a subject, comprising administering to the subject an effective therapeutic amount of a therapeutic agent formulated with a suitable pharmaceutical carrier, wherein the therapeutic agent is a crystallin protein, or fragment thereof.

2. The method of claim 1, wherein the crystallin protein, or fragment thereof, is selected from the group consisting of α crystallin, β crystallin, and a combination thereof.

3. The method of claim 1, wherein the subject is human.

4. The method of claim 1, wherein the administration is selected from the group consisting of intravenous injection, intramuscular injection, intraperitoneal injection, oral, subcutaneous, sublingual, and combinations thereof.

5. The method of claim 4, wherein the administration is intravenous injection.

6. The method of claim 2, wherein the crystalline protein, or fragment thereof, is αA crystallin.

7. The method of claim 2, wherein the crystalline protein, or fragment thereof, is β crystallin.

8. The method of claim 1, wherein the suitable carrier is saline.

9. The method of claim 1, wherein the effective therapeutic amount ranges from about 1 μg to about 20 μg of crystallin, or fragment thereof.

10. The method of claim 1, wherein further comprising treatment of retinitis pigmentosa, macular degeneration, diabetic retinopathy and/or glaucoma

11. A method for treating uveitis in a subject, comprising administering to the subject an effective therapeutic amount of a therapeutic agent formulated with a suitable pharmaceutical carrier, wherein the therapeutic agent is αA crystalline or fragment thereof.

12. The method of claim 11, wherein the subject is human.

13. The method of claim 11, wherein the administration is selected from the group consisting of intravenous injection, intramuscular injection, intraperitoneal injection, oral, subcutaneous, sublingual, and combinations thereof.

14. The method of claim 13, wherein the administration is intravenous injection.

15. The method of claim 11, wherein the suitable carrier is saline.

16. The method of claim 11, wherein the effective therapeutic amount ranges from about 1 μg to about 20 μg of crystallin, or fragment thereof.

17. A method for treating uveitis in a subject, comprising administering to the subject an effective therapeutic amount of a therapeutic agent formulated with a suitable pharmaceutical carrier, wherein the therapeutic agent is β crystallin, or fragment thereof.

18. The method of claim 17, wherein the subject is human.

19. The method of claim 17, wherein the administration is selected from the group consisting of intravenous injection, intramuscular injection, intraperitoneal injection, oral, subcutaneous, sublingual, and combinations thereof.

20. The method of claim 19, wherein the administration is intravenous injection.

21. The method of claim 17, wherein the suitable carrier is saline.

22. The method of claim 17, wherein the effective therapeutic amount ranges from about 1 μg to about 20 μg of crystalline or fragment thereof.
